(19) United States
(12) Patent Application Publication
(10) Pub. No.: US 2020/0096788 A1
(43) Pub. Date: Mar. 26, 2020

(54) DISCRETE SIGNAL PATHS FOR AN OPHTHALMIC DEVICE

(71) Applicant: Johnson & Johnson Vision Care, Inc., Jacksonville, FL (US)

(72) Inventors: Scott Humphreys, Greensboro, NC (US); Steven Hoggarth, Cary, NC (US); Adam Toner, Jacksonville, FL (US)

(21) Appl. No.: 16/138,258

(22) Filed: Sep. 21, 2018

(51) Int. Cl. G02C 7/04; G02C 7/08; G02B 1/04; A61B 5/00; A61B 3/10; A61B 5/145; G06K 9/00

(52) U.S. Cl. CPC G02C 7/049; G02C 7/081; G02B 1/043; A61F 9/0017; A61B 3/10; A61B 5/14507; G06K 9/00335; A61B 5/6821

(57) ABSTRACT

A discrete signal path of the present disclosure is able to distinguish between normal blink patterns and unique purposeful blinking patterns in order to control functionality in a powered ophthalmic lens. The discrete signal path of the present disclosure is able to detect the presence or absence of a non-human-capable communication sequence, such as a computer-generated communication signal of alternating light patterns that are unlikely to be accomplished by a human eye. The discrete signal path of the present disclosure is also able to be integrated into an ophthalmic device.

### BACKGROUND OF THE DISCLOSURE

### 1. Field of the Disclosure

The present disclosure relates to discrete signal paths configured to process input signals and more particularly, to ophthalmic devices, such as wearable lenses, including contact lenses, implantable lenses, including intraocular lenses (IDLs) and any other type of device comprising optical components that incorporate the discrete signal paths.

### 2. Discussion of the Related Art

As electronic devices continue to be miniaturized, it is becoming increasingly more likely to create wearable or embeddable microelectronic devices for a variety of uses. Such uses may include monitoring aspects of body chemistry, administering controlled dosages of medications or therapeutic agents via various mechanisms, including automatically, in response to measurements, or in response to external control signals, and augmenting the performance of organs or tissues. Examples of such devices include glucose infusion pumps, pacemakers, defibrillators, ventricular assist devices and neurostimulators. A new, particularly useful field of application is in ophthalmic wearable lenses and contact lenses. For example, a wearable lens may incorporate a lens assembly having an electronically adjustable focus to augment or enhance performance of the eye. In another example, either with or without adjustable focus, a wearable contact lens may incorporate electronic sensors to detect concentrations of particular chemicals in the precorneal (tear) film. The use of embedded electronics in a lens assembly introduces a potential requirement for communication with the electronics, for a method of powering and/or re-energizing the electronics, for interconnecting the electronics, for internal and external sensing and/or monitoring, and for control of the electronics and the overall function of the lens.

The human eye has the ability to discern millions of colors, adjust easily to shifting light conditions, and transmit signals or information to the brain at a rate exceeding that of a high-speed internet connection. Lenses, such as contact lenses and intraocular lenses, currently are utilized to correct vision defects such as myopia (nearsightedness), hyperopia (farsightedness), presbyopia and astigmatism. However, properly designed lenses incorporating additional components may be utilized to enhance vision as well as to correct vision defects.

Contact lenses may be utilized to correct myopia, hyperopia, astigmatism as well as other visual acuity defects. Contact lenses may also be utilized to enhance the natural appearance of the wearer's eyes. Contact lenses or “contacts” are simply lenses placed on the anterior surface of the eye. Contact lenses are considered medical devices and may be worn to correct vision and/or for cosmetic or other therapeutic reasons. Contact lenses have been utilized commercially to improve vision since the 1950s. Early contact lenses were made or fabricated from hard materials, were relatively expensive and fragile. In addition, these early contact lenses were fabricated from materials that did not allow sufficient oxygen transmission through the contact lens to the conjunctiva and cornea which potentially could cause a number of adverse clinical effects. Although these contact lenses are still utilized, they are not suitable for all patients due to their poor initial comfort. Later developments in the field gave rise to soft contact lenses, based upon hydrogels, which are extremely popular and widely utilized today. Specifically, silicone hydrogel contact lenses that are available today combine the benefit of silicone, which has extremely high oxygen permeability, with the proven comfort and clinical performance of hydrogels. Essentially, these silicone hydrogel based contact lenses have higher oxygen permeability and are generally more comfortable to wear than the contact lenses made of the earlier hard materials.

Conventional contact lenses are polymeric structures with specific shapes to correct various vision problems as briefly set forth above. To achieve enhanced functionality, various circuits and components have to be integrated into these polymeric structures. For example, control circuits, microprocessors, communication devices, power supplies, sensors, actuators, light-emitting diodes, and miniature antennas may be integrated into contact lenses via custom-built optoelectronic components to not only correct vision, but to enhance vision as well as provide additional functionality as is explained herein. Electronic and/or powered contract lenses may be designed to provide enhanced vision via zoom-in and zoom-out capabilities, or just simply modifying the refractive capabilities of the lenses. Electronic and/or powered contact lenses may be designed to enhance color and resolution, to display textural information, to translate speech into captions in real time, to offer visual cues from a navigation system, and to provide image processing and internet access. The lenses may be designed to allow the wearer to see in low-light conditions. The properly designed electronics and/or arrangement of electronics on lenses may allow for projecting an image onto the retina, for example, without a variable-focus optic lens, provide novelty image displays and even provide wakeup alerts. Alternately, or in addition to any of these functions or similar functions, the contact lenses may incorporate components for the noninvasive monitoring of the wearer's biomarkers and health indicators. For example, sensors built into the lenses may allow a diabetic patient to keep tabs on blood sugar levels by analyzing components of the tear film without the need for drawing blood. In addition, an appropriately configured lens may incorporate sensors for monitoring cholesterol, sodium, and potassium levels, as well as other biological markers. This, coupled with a wireless data transmitter, could allow a physician to have almost immediate access to a patient's blood chemistry without the need for the patient to waste time getting to a laboratory and having blood drawn. In addition, sensors built into the lenses may be utilized to detect light incident on the eye to compensate for ambient light conditions or for use in determining blink patterns.

The proper combination of devices could yield potentially unlimited functionality; however, there are a number of difficulties associated with the incorporation of extra components on a piece of optical-grade polymer. In general, it is difficult to manufacture such components directly on the lens for a number of reasons, as well as mounting and interconnecting planar devices on a non-planar surface. It is also difficult to manufacture to scale. The components to be placed on or in the lens need to be miniaturized and integrated onto just 1.5 square centimeters of a transparent polymer while protecting the components from the liquid environment on the eye. It is also difficult to make a contact lens comfortable and safe for the wearer with the added thickness of additional components.

Given the area and volume constraints of an ophthalmic device such as an intraocular device or contact lens, and the environment in which it is to be utilized, the physical realization of the device must overcome a number of problems, including mounting and interconnecting a number of electronic components on a non-planar surface, the bulk of which comprises optic plastic. Accordingly, there exists a need for providing mechanically and electrically robust electronic ophthalmic devices.

As these are powered devices (e.g., lenses), energy or more particularly current consumption, to run the electronics is a concern given battery technology on the scale for an ophthalmic lens. In addition to normal current consumption, powered devices or systems of this nature generally require standby current reserves, precise voltage control and switching capabilities to ensure operation over a potentially wide range of operating parameters, and burst consumption, for example, up to eighteen (18) hours on a single charge, after potentially remaining idle for years. Accordingly, there exists a need for devices and systems that are optimized for low-cost, long-term reliable service, safety and size while providing the required power.

Powered or electronic ophthalmic devices such as lenses may employ ambient or infrared light sensors to detect ambient lighting conditions, blinking by the wearer, and/or visible or infrared communication signals from another device. Blink detection or light-based communication may be utilized as a means to control one or more aspects of a powered ophthalmic lens. Additionally, external factors, such as changes in light intensity levels, and the amount of visible light that a person's eyelid blocks out, have to be accounted for when determining blinks. As an example, a photosensor system may be sensitive enough to detect light intensity changes that occur when a person blinks over a wide range of lighting conditions. For example, a typical room has an illumination level between fifty (50) and three hundred (300) lux, while illumination levels out of doors may be between five hundred (500) and fifty thousand (50,000) lux depending on time of day and cloud cover.

Powered or electronic ophthalmic lenses may need to respond to additional or more specific command or control signals provided by a transmitter operated by the individual wearer or another individual such as a clinician. Communication receivers impose design constraints on power consumption, area and volume. The receiver may conserve power by periodically turning on (waking-up or strobing) and searching for a transmission. Accordingly there is a need for discrete signal paths for receiving and processing signals that minimize complexity, power consumption, area and volume to a powered or electronic ophthalmic lens.

### SUMMARY OF THE DISCLOSURE

The electronic ophthalmic devices and discrete signal paths in accordance with the present disclosure overcome one or more of the limitations associated with the prior art as briefly described above.

The present disclosure relates to powered ophthalmic devices comprising an electronic system, which performs any number of functions, including actuating a variable-focus optic if included. The electronic system includes one or more batteries or other power sources, power management circuitry, one or more sensors, clock generation circuitry, control algorithms, circuitry comprising a discrete signal path, and lens driver circuitry.

The discrete signal paths of the present disclosure are, in one aspect, able to distinguish between normal blink patterns and unique purposeful blinking patterns in order to control functionality in a powered ophthalmic lens. The discrete signal paths of the present disclosure are able to detect the presence or absence of a non-human-capable communication sequence, such as a communication sequence of alternating light patterns that are unlikely to be accomplished by a human eye. The discrete signal paths of the present disclosure are also able to be integrated into a contact lens, for example, as part of an electronic system. As a further example, each of the discrete signal paths may comprise one or more of a photodetector, a signal processing block, and a sequence detector, as described herein. Other components may be included in accordance various aspects of the disclosure.

In accordance with one aspect, the present disclosure is directed to methods for detecting signal patterns, which methods may include sampling, via a first signal path disposed on an ophthalmic device, light incident on an eye of an individual and at least temporarily saving first collected samples; sampling, via a second signal path disposed on an ophthalmic device, light incident on an eye of an individual and at least temporarily saving second collected samples, wherein the second signal path is discrete from the first signal path; analyzing the first collected samples to determine the existence or absence of a human-capable blink pattern; analyzing the second collected samples to determine the existence or absence of a non-human-capable communication sequence; and providing an indication signal to activate and control one or more properties of the ophthalmic device based at least on one or more of the existence or absence of the human-capable blink pattern and the existence or absence of the non-human-capable communication sequence.

In accordance with another aspect, the present disclosure is directed to methods for detecting signal patterns, which methods may include sampling, via a first signal path disposed on an ophthalmic device that fits on or in an eye of a user, light incident on an eye of an individual and at least temporarily saving collected samples; analyzing, by a controller and via the first signal path, the collected samples to determine the existence or absence of a human-capable blink pattern; energizing (e.g., or triggering), based on the absence of a human-capable blink pattern, a second signal to enable analysis of the collected samples to determine a sequence indicative of an embedded communication message; and providing an indication signal to a control system to activate and control one or more properties of the ophthalmic device based at least on the embedded communication message.

The discrete signal path may be in communication with detection logic such as a sequence detector, which may be configured to detect characteristics of blinks (e.g., human blinks or non-human communication patterns), for example, if the lid is open or closed, the duration of the blink open or closed, the inter-blink duration, and the number of blinks in a given time period. An exemplary algorithm in accordance with the present disclosure relies on sampling light incident on the eye at a certain sample rate. Pre-determined blink patterns are stored and compared to the recent history of incident light samples. When patterns match, the blink detection algorithm may trigger activity in a system controller, for example, to activate the lens driver to change the refractive power of the lens.

The discrete signal paths and associated circuitry of the present disclosure preferably operate over a reasonably wide range of lighting conditions and is preferably able to distinguish an intentional blink sequence from involuntary blinks. The discrete signal paths and associated circuitry provide a safe, low cost, and reliable means and method for detecting blinks via a powered or electronic ophthalmic device, which also has a low rate of power consumption and is scalable for incorporation into an ophthalmic lens, for at least one of activating or controlling a powered or electronic ophthalmic lens.

In accordance with one aspect, the present disclosure is directed to powered ophthalmic devices comprising an electronic system. The electronic systems comprise a photodetector comprising one or more photodiodes producing an output current, a signal processing circuit comprising electronic circuits and receiving the output current and providing an output signal based on the output current, and a system controller receiving the output signal, wherein the system controller is configured to detect one or more blink sequences and a non-human-capable communication sequence (e.g., special IR sequence, data sequence, embedded data message, etc.), and wherein the photodetector and signal processing circuit substantially utilize the same photodiodes and circuitry to receive and process the one or more blink sequences and non-human-capable communication sequences, thereby minimizing additional complexity, power consumption area and volume to support both blink detection and an infrared communication signal reception.

### BRIEF DESCRIPTION OF THE DRAWINGS

The foregoing and other features and advantages of the disclosure will be apparent from the following, more particular description of preferred embodiments of the disclosure, as illustrated in the accompanying drawings.

FIG. 1 illustrates an exemplary ophthalmic lens comprising a blink detection and communication system having discrete signal paths in accordance with some embodiments of the present disclosure.

FIG. 2 illustrates a photodetector system in accordance with some embodiments of the present disclosure.

FIG. 3 is a block diagram of digital detection logic in accordance with some embodiments of the present disclosure.

FIG. 4 illustrates human-capable blink and non-human-capable communication sequences in accordance with some embodiments of the present disclosure.

FIG. 5 illustrates a timing diagram of an operational sequence of a combined blink detection and communication system in accordance with some embodiments of the present disclosure.

FIG. 6 is a diagrammatic representation of an exemplary electronic insert, including a combined blink detection and communication system, positioned in a powered or electronic ophthalmic device in accordance with the present disclosure.

### DETAILED DESCRIPTION OF THE PREFERRED EMBODIMENTS

Conventional contact lenses are polymeric structures with specific shapes to correct various vision problems as briefly set forth above. To achieve enhanced functionality, various circuits and components may be integrated into these polymeric structures. For example, control circuits, microprocessors, communication devices, power supplies, sensors, actuators, light-emitting diodes, and miniature antennas may be integrated into contact lenses via custom-built optoelectronic components to not only correct vision, but to enhance vision as well as provide additional functionality as is explained herein. Electronic and/or powered contact lenses may be designed to provide enhanced vision via zoom-in and zoom-out capabilities, or just simply modifying the refractive capabilities of the lenses. Electronic and/or powered contact lenses may be designed to enhance color and resolution, to display textural information, to translate speech into captions in real time, to offer visual cues from a navigation system, and to provide image processing and internet access. The lenses may be designed to allow the wearer to see in low light conditions. The properly designed electronics and/or arrangement of electronics on lenses may allow for projecting an image onto the retina, for example, without a variable focus optic lens, provide novelty image displays and even provide wakeup alerts. Alternately, or in addition to any of these functions or similar functions, the contact lenses may incorporate components for the noninvasive monitoring of the wearer's biomarkers and health indicators. For example, sensors built into the lenses may allow a diabetic patient to keep tabs on blood sugar levels by analyzing components of the tear film without the need for drawing blood. In addition, an appropriately configured lens may incorporate sensors for monitoring cholesterol, sodium, and potassium levels, as well as other biological markers. This coupled with a wireless data transmitter could allow a physician to have almost immediate access to a patient's blood chemistry without the need for the patient to waste time getting to a laboratory and having blood drawn. In addition, sensors built into the lenses may be utilized to detect light incident on the eye to compensate for ambient light conditions or for use in determining blink patterns.

The powered or electronic ophthalmic device of the present disclosure comprises the necessary elements to correct and/or enhance the vision of patients with one or more of the above described vision defects or otherwise perform a useful ophthalmic function. In addition, the electronic ophthalmic device may be utilized simply to enhance normal vision or provide a wide variety of functionality as described above. The electronic ophthalmic device may comprise a variable focus optic lens, an assembled front optic embedded into a contact lens or just simply embedding electronics without a lens for any suitable functionality. The electronic lens of the present disclosure may be incorporated into any number of contact lenses as described above. In addition, intraocular lenses may also incorporate the various components and functionality described herein. However, for ease of explanation, the disclosure will focus on an electronic ophthalmic device to correct vision defects intended for single-use daily disposability.

Throughout the specification the terms ophthalmic device and ophthalmic device are utilized. In general terms, an ophthalmic device may include contact lenses, intraocular lenses, spectacle lenses and punctal plugs. However, in accordance with the present disclosure, an ophthalmic device is one for eye disease treatment, vision correction and/or enhancement and preferably includes at least one of punctal plugs, spectacle lenses, contact lenses and intraocular lenses. An intraocular lens or IOL is a lens that is implanted in the eye and replaces the crystalline lens. It may be utilized for individuals with cataracts or simply to treat various refractive errors. An IOL typically comprises a small plastic lens with plastic side struts called haptics to hold the lens in position within the capsular bag in the eye. Any of the electronics and/or components described herein may be incorporated into IOLs in a manner similar to that of contact lenses. A punctal plug or occluder is an ophthalmic device for insertion into a punctum of an eye in order to treat one or more disease states. While the present disclosure may be utilized in any of these devices, in preferred exemplary embodiments, the present disclosure is utilized in contact lenses or intraocular lenses.

The present disclosure may be employed in a powered ophthalmic lens or powered contact lens comprising an electronic system, which actuates a variable-focus optic or any other device or devices configured to implement any number of numerous functions that may be performed. The electronic system includes one or more batteries or other power sources, power management circuitry, one or more sensors, clock generation circuitry, control algorithms and circuitry, and lens driver circuitry. The complexity of these components may vary depending on the required or desired functionality of the lens.

Control of an electronic or a powered ophthalmic lens may be accomplished through a manually operated external device that communicates with the lens, such as a hand-held remote unit. For example, a fob may wirelessly communicate with the powered lens based upon manual input from the wearer. Alternately, control of the powered ophthalmic lens may be accomplished via feedback or control signals directly from the wearer. For example, sensors built into the lens may detect blinks and/or blink patterns. Based upon the pattern or sequence of blinks, the powered ophthalmic lens may change state, for example, its refractive power in order to either focus on a near object or a distant object. As a further example, sensors built into the lens may detect non-human-capable light patterns or sequences such as generated light communications caused to be incident on a wearer's eye. Based upon the pattern or sequence represented in the light communication, the powered ophthalmic lens may execute an operation.

Additionally or alternately, blink detection in a powered or electronic ophthalmic lens may be used for other various uses where there is interaction between the user and the electronic ophthalmic device, such as activating another electronic device, or sending a command to another electronic device. For example, blink detection in an ophthalmic lens may be used in conjunction with a camera on a computer wherein the camera keeps track of where the eye(s) moves on the computer screen, and when the user executes a blink sequence that it detected, it causes the mouse pointer to perform a command, such as double-clicking on an item, highlighting an item, or selecting a menu item.

A blink detection algorithm is a component of the system controller which detects characteristics of blinks, for example, is the lid open or closed, the duration of the blink, the inter-blink duration, and the number of blinks in a given time period. One algorithm in accordance with the present disclosure relies on sampling light incident on the eye at a certain sample rate. Pre-determined blink patterns may be stored and compared to the recent history of incident light samples. When patterns match, the blink detection algorithm may trigger activity in the system controller, for example, to activate the lens driver to change the refractive power of the lens.

Blinking is the rapid closing and opening of the eyelids and is an essential function of the eye. Blinking protects the eye from foreign objects, for example, individuals blink when objects unexpectedly appear in proximity to the eye. Blinking provides lubrication over the anterior surface of the eye by spreading tears. Blinking also serves to remove contaminants and/or irritants from the eye. Normally, blinking is done automatically, but external stimuli may contribute as in the case with irritants. However, blinking may also be purposeful, for example, for individuals who are unable to communicate verbally or with gestures can blink once for yes and twice for no. The blink detection algorithm and system of the present disclosure utilizes blinking patterns that cannot be confused with normal blinking response. In other words, if blinking is to be utilized as a means for controlling an action, then the particular pattern selected for a given action cannot occur at random; otherwise inadvertent actions may occur. As blink speed may be affected by a number of factors, including fatigue, eye injury, medication and disease, blinking patterns for control purposes preferably account for these and any other variables that affect blinking. The average length of involuntary blinks is in the range of about one hundred (100) to four hundred (400) milliseconds. Average adult men and women blink at a rate of ten (10) involuntary blinks per minute, and the average time between involuntary blinks is about 0.3 to seventy (70) seconds.

An exemplary embodiment of a blink detection algorithm may be summarized in the following steps.

1. Define an “blink sequence” (e.g., intentional or unintentional blink sequence) that a user will execute for positive blink detection.

2. Sample the incoming light level at a rate consistent with detecting the blink sequence and rejecting involuntary blinks.

3. Compare the history of sampled light levels to the expected “blink sequence,” as defined by a blink template of values.

4. Optionally implement a blink “mask” sequence to indicate portions of the template to be ignored during comparisons, e.g. near transitions. This may allow for a user to deviate from a desired “blink sequence,” such as a plus or minus one (1) error window, wherein one or more of lens activation, control, and focus change can occur.

Additionally, this may allow for variation in the user's timing of the blink sequence.

An exemplary blink sequence may be defined as follows:

1. blink (closed) for 0.5 s

2. open for 0.5 s

3. blink (closed) for 0.5 s

At a one hundred (100) ms sample rate, a twenty (20) sample blink template is given by

blink_template=[1, 1, 1, 0, 0, 0, 0, 0, 1, 1, 1, 1, 1, 0, 0, 0, 0, 0, 1, 1].

The blink mask is defined to mask out the samples just after a transition (0 to mask out or ignore samples), and is given by

blink_mask=[1, 1, 1, 0, 1, 1, 1, 1, 0, 1, 1, 1, 1, 0, 1, 1, 1, 1, 0, 1].

Optionally, a wider transition region may be masked out to allow for more timing uncertainty, and is given by

blink_mask=[1, 1, 0, 0, 1, 1, 1, 0, 0, 1, 1, 1, 0, 0, 1, 1, 1, 0, 0, 1].

Alternate patterns may be implemented, e.g. single long blink, in this case a 1.5 s blink with a 24-sample template, given by

blink_template=[1, 1, 1, 1, 0, 0, 0, 0, 0, 0, 0, 0, 0, 0, 0, 0, 0, 0, 0, 1, 1, 1, 1, 1].

It is important to note that the above example is for illustrative purposes and does not represent a specific set of data.

Detection may be implemented by logically comparing the history of samples against the template and mask. The logical operation is to exclusive-OR (XOR) the template and the sample history sequence, on a bitwise basis, and then verify that all unmasked history bits match the template. For example, as illustrated in the blink mask samples above, in each place of the sequence of a blink mask that the value is logic 1, a blink has to match the blink mask template in that place of the sequence. However, in each place of the sequence of a blink mask that the value is logic 0, it is not necessary that a blink matches the blink mask template in that place of the sequence. For example, the following Boolean algorithm equation, as coded in MATLAB®, may be utilized:

matched=not(blink_mask)|not(xor(blink_template,test_sample)),

wherein test_sample is the sample history. The matched value is a sequence with the same length as the blink template, sample history and blink mask. If the matched sequence is all logic 1's, then a good match has occurred. Breaking it down, not (xor (blink_template, test_sample)) gives a logic 0 for each mismatch and a logic 1 for each match. Logic oring with the inverted mask forces each location in the matched sequence to a logic 1 where the mask is a logic 0. Accordingly, the more places in a blink mask template where the value is specified as logic 0, the greater the margin of error in relation to a person's blinks is allowed. MATLAB® is a high level language and implementation for numerical computation, visualization and programming and is a product of MathWorks, Natick, Mass. It is also important to note that the greater the number of logic 0's in the blink mask template, the greater the potential for false positive matched to expected or intended blink patterns. Additionally or alternatively, pseudo code for may be implemented, such as:

match if (mask & (template\hat{ }history)==0)

where & is a bitwise AND, \hat{ } is bitwise XOR and ==0 tests whether the value of the result equals zero.

It should be appreciated that a variety of expected or intended blink patterns may be programmed into a device with one or more active at a time. More specifically, multiple expected or intended blink patterns may be utilized for the same purpose or functionality, or to implement different or alternate functionality. For example, one blink pattern may be utilized to cause the lens to zoom in or out on an intended object while another blink pattern may be utilized to cause another device, for example, a pump, on the lens to deliver a dose of a therapeutic agent.

Additionally or alternatively, the signal processing path configured to implement a blink detection algorithm, as described herein, may be configured to detect non-human-capable light sequences or patterns (e.g., non-human-capable communication sequence) such as computer-generated communication signals. For example, a special light sequence may define at least a portion of a non-human-capable communication sequence and may be caused to be transmitted to an eye of a wearer and may represent a pattern of alternating high and low light levels that has a frequency beyond a human-capable threshold for blinking. In some embodiments the special light sequence may comprise a number of, for example six, alternating high and low intervals of 0.2 seconds each. Such a sequence would be very unlikely to be produced by a human eye lid, and thus represents a unique sequence not produced by blinking. The special light sequence may be a programmable sequence and may be used as a trigger signal or preamble to indicate presence of or starting of an embedded data message. Although the term “non-human-capable” is used to differentiate signals from those that may be attributed to typical human-capable blink patterns, such non-human-capable sequences may be any pattern. Such non-human-capable sequences may have a frequency, duration, and/or complexity that is pre-defined to distinguish itself from human-capable blink patterns. As such, the systems described herein may be configured to determine the presence or absence of a human-capable blink pattern and a non-human-capable communication sequence using the same discrete processing path.

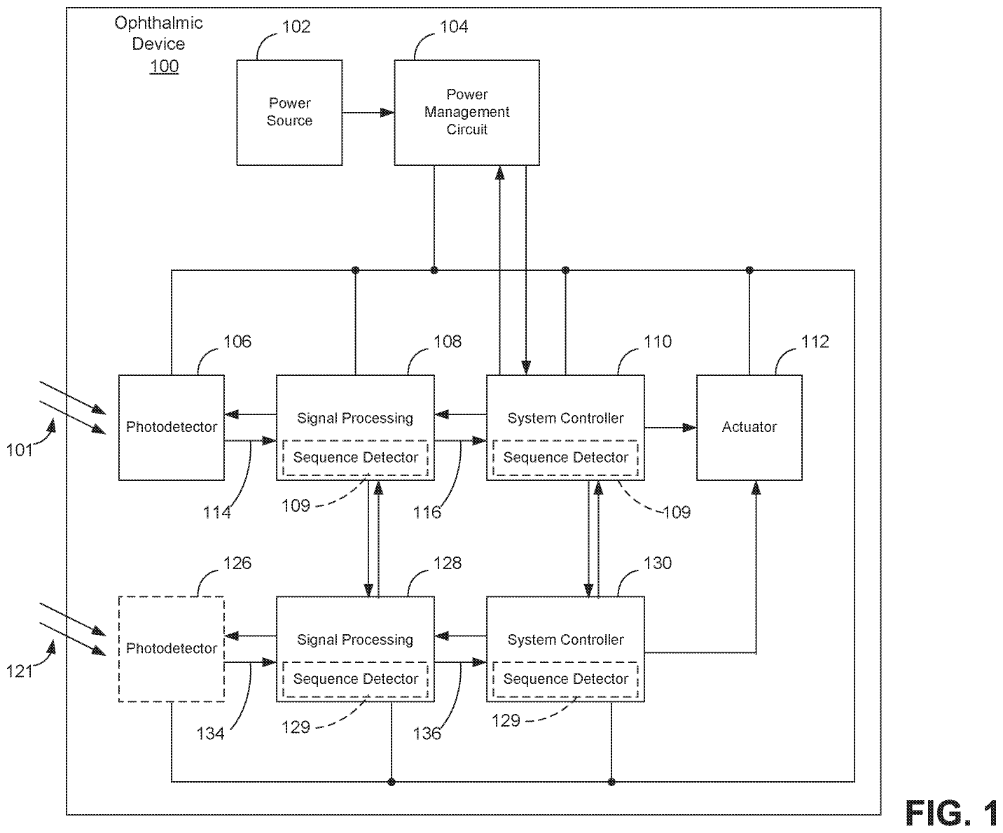

FIG. 1 illustrates, in block diagram form, an exemplary powered ophthalmic device 100 (e.g., lens) comprising a multipath blink detection and communication system. The ophthalmic lens 100 may include a power source 102 and a power management circuit 104 configured to provide electrical energy to other components of the ophthalmic device 100 and to manage the electrical characteristics of the energy provide. As an example, the power management circuit 104 may be configured to provide particular current to or potential across various devices. As a further example, the power management circuit 104 may be configured to selectively energize (e.g., or trigger) and de-energize various components and or groups of components (e.g., processing paths). Such selective energizing of components may conserve energy and may allow certain processing on an as-needed basis, as described herein.

The ophthalmic lens 100 may include a photodetector 106, a signal processing circuit 108 or block, a system controller 110 and an actuator 112. As an example, one or more of the photodetector 106, the signal processing circuit 108 or block, and the system controller 110 may define a first signal path. When the ophthalmic lens 100 is placed onto the front surface of a user's eye the photodetector 106, the signal processing circuit 108, and the system controller 110 may be utilized to detect ambient light, variation in incident light levels, and/or infrared communication signals and may be utilized to control the actuator 112. Although FIG. 1 illustrates an example of an ophthalmic lens, the components and circuitry described herein may be applied to other and ophthalmic devices, such as wearable lenses, including contact lenses, implantable lenses, including intraocular lenses (IDLs) and any other type of device comprising optical components that incorporate electronic circuits and associated signal paths configured to process one or more inputs received by the ophthalmic device.

The photodetector 106 may be embedded into the ophthalmic lens 100. As such, the photodetector 106 may be configured to receive light such as ambient or infrared light 101 that is incident to the ophthalmic lens 100 and/or eye of a wearer of the ophthalmic lens 100. The photodetector 106 may be configured to generate and/or transmit a light-based signal 134 having a value representative of the light energy incident on the ophthalmic lens 100. As an example, the light-based signal 134 may be provided to the signal processing circuit 108 or other processing mechanism. The photodetector 106 and the signal processing circuit 108 may define at least a portion of the discrete signal path, as described herein.

The photodetector 106 and the signal processing circuit 108 may be configured for two-way communication. The signal processing circuit 108 may provide one or more signals to the photodetector 106, examples of which are set forth subsequently. The signal processing circuit 108 may include circuits configured to perform analog to digital conversion and digital signal processing, including one or more of filtering, processing, detecting, and otherwise manipulating/processing data to permit incident light detection for downstream use. As an example, the signal processing circuit 108 may be configured to effect signal conversion such as current or charge to voltage, analog-to-digital (analog-to-digital converter/conversion (ADC). As another example, the signal processing circuit 108 may be configured to provide ADC control such as peak/valley/threshold generation, data slicing, and automatic gain control (AGC). Other components and functions may be included.

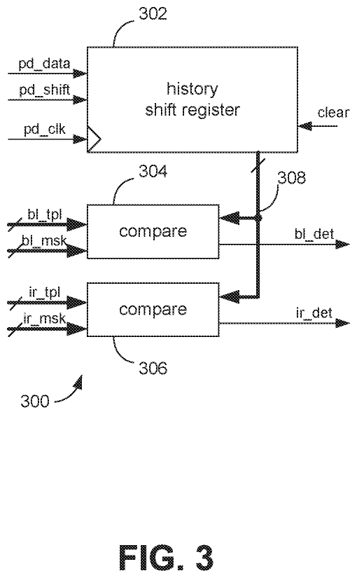

The signal processing circuit 108 may provide a data signal 116 based on the light-based signal 134. As an example, the data signal 116 may be provided to a sequence detector 109. For example, a sequence detector 109 may be configured to detect and analyze input signal to determine the existence or absence of certain sequences. The sequence detector 109 may include digital detection logic (e.g., logic 300 (FIG. 3)), as described in further detail below. The sequence detector 109 may be configured as part of the signal processing circuit 108 and/or the system controller 110. Additionally or alternatively, the sequence detector 109 may be configured separately from the signal processing circuit 108 and/or the system controller 110.

The system controller 110 and the signal processing circuit 108 may be configured for two-way communication. The system controller 110 may provide one or more control or data signals to the signal processing circuit 108, examples of which are set forth subsequently. The system controller 110 may be configured to detect sequences of light variation indicative of specific blink patterns or infrared communication protocols, for example, via the sequence detector 109. Upon detection of a sequence, the system controller 110 may act or may be caused to act to change the state of actuator 112, for example, by enabling, disabling or changing an operating parameter such as an amplitude or duty cycle of the actuator 112. In certain embodiments, the system controller 110 may comprise components such as a digital receiver configured to process signals and to extract information such as sync words, device addresses, messages, and the like. In certain embodiments, the system controller 110 may comprise components such as a state machine or master controller configures to change the state of one or more systems or components. Other configurations of the system controller 110 may be used to effect change of the actuator 112 and/or other components.

As an illustrative example, the sequence detector 109 may be configured to detect sequences of light variation indicative of a human-capable pattern or sequence such as a blink pattern. In some embodiments the blink sequence may comprise two low intervals of 0.5 seconds separated by a high interval of 0.5 seconds. A template of length 24 of data values representative of the blink sequence sampled at a 0.1 second or 10 Hz rate is [1, 1, 1, 1, 1, 1, 0, 0, 0, 0, 0, 1, 1, 1, 1, 1, 0, 0, 0, 0, 0, 1, 1, 1].

As described herein, the first signal path (e.g., sequence detector 109) may be configured to detect the presence or absence of sequences of light variation indicative of a human-capable pattern or sequence such as a blink pattern and a second signal path (e.g., sequence detector 129) may be configured to detect sequences of light variation indicative of a non-human-capable pattern or sequence.

The sequence detector 109 may be configured to detect sequences of light variation indicative of a non-human-capable pattern or sequence such as a generated infrared communication signal. In some embodiments, a non-human-capable communication sequence (e.g., IR sequence) may comprise a number of, for example six, alternating high and low intervals of 0.2 seconds each. Such a sequence would be very unlikely to be produced by a human eye lid, and thus represents a unique sequence not produced by blinking. In the present disclosure the special IR sequence indicates that a higher data rate IR data message is starting or is present. A template of length 24 of data values representative of the IR sequence sampled at a 0.1 second or 10 Hz rate is [1, 1, 0, 0, 1, 1, 0, 0, 1, 1, 0, 0, 1, 1, 0, 0, 1, 1, 0, 0, 1, 1, 0, 0].

The signal processing circuit 108 may provide (or cause provision of) an indication signal to the photodetector 106 to automatically adjust the gain of the photodetector 106 in response to ambient or received light levels in order to maximize the dynamic range of the system. The system controller 110 may provide one or more control signals to the signal processing circuit 108 to initiate a data conversion operation or to enable or disable automatic gain adjustment of the photodetector 106 and signal processing circuit 108 in different modes of operation. The system controller 110 may be configured to periodically enable the photodetector 106 and the signal processing circuit 108 to periodically sample the light 101. The system controller 110 may be further configured to modify the sample rate depending on a mode of operation. For example, a low sample rate may be used for detection of a blink sequence or an IR sequence, and a high sample rate may be used for receiving and decoding an infrared communication signal (e.g., data message) having a higher data rate or symbol rate than may be accommodated with the low sample rate. For example, a low sample rate of 0.1 s per sample or 10 Hz may be used for detection of the sequences, and a high sample rate of 390.625 us per sample or 2.56 kHz may be used for sampling of an infrared communication signal having a symbol rate of 3.125 ms per symbol or 320 symbols per second.

Automatic gain control systems as described above may have one or more associated time constants corresponding to the response time of the automatic gain control functions. In order to minimize complexity of the combined blink detection and communication system the automatic gain control system of the signal processing circuit 108 may be optimized for operation during detection of blink sequences and not for higher data rate communication signals (e.g., data message). In this case the system controller 110 may disable the automatic gain control system and further may direct the signal processing circuit 108 to hold the gain at a high level when operating with a high sample rate. For example, some embodiments of the powered ophthalmic lens 100 may support infrared signal detection only in environments with ambient light levels below 5000 lux and with infrared communication signals having incident power greater than 1 watt per square meter. The signal processing circuit 108 may operate with a gain dependent on the sample rate, an example of which is set forth subsequently. Under this range of conditions it may be possible to provide the data signal 116 with sufficient signal-to-noise ratio for detection while configuring the photodetector 106 and signal processing circuit 108 to have a constant gain from incident light energy to the amplitude or value of the data signal 116. In this way the system complexity may be minimized compared to a system that may operate with variable gain during infrared communication signal detection or processing.

In some embodiments, the signal processing circuit 108 may define at least a portion of the discrete signal path, as described herein. The signal processing circuit 108 may be implemented as a system comprising an integrating sampler, an analog to digital converter and a digital logic circuit configured to provide a digital data signal 116 based on the light-based signal 114. The system controller 110 also may be implemented as a digital logic circuit and implemented as a separate component or integrated with signal processing circuit 108. Portions of the signal processing circuit 108 and system controller 110 may be implemented in custom logic, reprogrammable logic or one or more microcontrollers as are well known to those of ordinary skill in the art. The signal processing circuit 108 and system controller 110 may comprise associated memory to maintain a history of values of the light-based signal 114, the data signal 116 or the state of the system. Any suitable arrangement and/or configuration may be utilized.

The ophthalmic lens 100 may include a photodetector 126, a signal processing circuit 128 or block, a system controller 130. As an example, one or more of the photodetector 126, the signal processing circuit 128 or block, and the system controller 130 may define a second signal path. In certain aspects, the second signal path may be configured to receive data and/or samples that were captured via the photodetector 106, which may be part of the first signal path. As such, the single paths may share samples that were collected via the same photodetector or array.

When the ophthalmic lens 100 is placed onto the front surface of a user's eye the photodetector 126, the signal processing circuit 108, and the system controller 130 may be utilized to detect ambient light, variation in incident light levels, and/or infrared communication signals and may be utilized to control the actuator 112. Although FIG. 1 illustrates an example of an ophthalmic lens, the components and circuitry described herein may be applied to other and ophthalmic devices, such as wearable lenses, including contact lenses, implantable lenses, including intraocular lenses (IDLs) and any other type of device comprising optical components that incorporate electronic circuits and associated signal paths configured to process one or more inputs received by the ophthalmic device.

The photodetector 126 may be embedded into the ophthalmic lens 100. As such, the photodetector 126 may be configured to receive light such as ambient or infrared light 121 that is incident to the ophthalmic lens 100 and/or eye of a wearer of the ophthalmic lens 100. The photodetector 126 may be configured to generate and/or transmit a light-based signal 114 having a value representative of the light energy incident on the ophthalmic lens 100. As an example, the light-based signal 114 may be provided to the signal processing circuit 128 or other processing mechanism. The photodetector 126 and the signal processing circuit 128 may define at least a portion of the discrete signal path, as described herein.

The photodetector 126 and the signal processing circuit 128 may be configured for two-way communication. The signal processing circuit 128 may provide one or more signals to the photodetector 126, examples of which are set forth subsequently. The signal processing circuit 128 may include circuits configured to perform analog to digital conversion and digital signal processing, including one or more of filtering, processing, detecting, and otherwise manipulating/processing data to permit incident light detection for downstream use. As an example, the signal processing circuit 128 may be configured to effect signal conversion such as current or charge to voltage, analog-to-digital (analog-to-digital converter/conversion (ADC). As another example, the signal processing circuit 128 may be configured to provide ADC control such as peak/valley/threshold generation, data slicing, and automatic gain control (AGC). Other components and functions may be included.

The signal processing circuit 128 may provide a data signal 136 based on the light-based signal 134. As an example, the data signal 136 may be provided to a sequence detector 129. For example, a sequence detector 129 may be configured to detect and analyze input signal to determine the existence or absence of certain sequences. The sequence detector 129 may include digital detection logic (e.g., logic 300 (FIG. 3)), as described in further detail below. The sequence detector 129 may be configured as part of the signal processing circuit 128 and/or the system controller 130. Additionally or alternatively, the sequence detector 129 may be configured separately from the signal processing circuit 128 and/or the system controller 130.

The system controller 130 and the signal processing circuit 128 may be configured for two-way communication. The system controller 130 may provide one or more control or data signals to the signal processing circuit 128, examples of which are set forth subsequently. The system controller 130 may be configured to detect sequences of light variation indicative of specific blink patterns or infrared communication protocols, for example, via the sequence detector 129. Upon detection of a sequence, the system controller 130 may act or may be caused to act to change the state of actuator 112, for example, by enabling, disabling or changing an operating parameter such as an amplitude or duty cycle of the actuator 112. In certain embodiments, the system controller 130 may comprise components such as a digital receiver configured to process signals and to extract information such as sync words, device addresses, messages, and the like. In certain embodiments, the system controller 130 may comprise components such as a state machine or master controller configures to change the state of one or more systems or components. Other configurations of the system controller 130 may be used to effect change of the actuator 112 and/or other components.

As an illustrative example, the sequence detector 129 may be configured to detect sequences of light variation indicative of a human-capable pattern or sequence such as a blink pattern. In some embodiments the blink sequence may comprise two low intervals of 0.5 seconds separated by a high interval of 0.5 seconds. A template of length 24 of data values representative of the blink sequence sampled at a 0.1 second or 10 Hz rate is [1, 1, 1, 1, 1, 1, 0, 0, 0, 0, 0, 1, 1, 1, 1, 1, 0, 0, 0, 0, 0, 1, 1, 1].

The sequence detector 129 may be configured to detect sequences of light variation indicative of a non-human-capable pattern or sequence such as a generated infrared communication signal. In some embodiments, a non-human-capable communication sequence (e.g., IR sequence) may comprise a number of, for example six, alternating high and low intervals of 0.2 seconds each. Such a sequence would be very unlikely to be produced by a human eye lid, and thus represents a unique sequence not produced by blinking. In the present disclosure the special IR sequence indicates that a higher data rate IR data message is starting or is present. A template of length 24 of data values representative of the IR sequence sampled at a 0.1 second or 10 Hz rate is [1, 1, 0, 0, 1, 1, 0, 0, 1, 1, 0, 0, 1, 1, 0, 0, 1, 1, 0, 0, 1, 1, 0, 0].

As described herein, the first signal path (e.g., sequence detector 109) may be configured to detect the presence or absence of sequences of light variation indicative of a human-capable pattern or sequence such as a blink pattern and the second signal path (e.g., sequence detector 129) may be configured to detect sequences of light variation indicative of a non-human-capable pattern or sequence.

The signal processing circuit 128 may provide (or cause provision of) an indication signal to the photodetector 126 to automatically adjust the gain of the photodetector 126 in response to ambient or received light levels in order to maximize the dynamic range of the system. The system controller 130 may provide one or more control signals to the signal processing circuit 128 to initiate a data conversion operation or to enable or disable automatic gain adjustment of the photodetector 126 and signal processing circuit 128 in different modes of operation. The system controller 130 may be configured to periodically enable the photodetector 126 and the signal processing circuit 128 to periodically sample the light 121. The system controller 130 may be further configured to modify the sample rate depending on a mode of operation. For example, a low sample rate may be used for detection of a blink sequence or an IR sequence, and a high sample rate may be used for receiving and decoding an infrared communication signal (e.g., data message) having a higher data rate or symbol rate than may be accommodated with the low sample rate. For example, a low sample rate of 0.1 s per sample or 10 Hz may be used for detection of the sequences, and a high sample rate of 390.625 us per sample or 2.56 kHz may be used for sampling of an infrared communication signal having a symbol rate of 3.125 ms per symbol or 320 symbols per second.

Automatic gain control systems as described above may have one or more associated time constants corresponding to the response time of the automatic gain control functions. In order to minimize complexity of the combined blink detection and communication system the automatic gain control system of the signal processing circuit 128 may be optimized for operation during detection of blink sequences and not for higher data rate communication signals (e.g., data message). In this case the system controller 130 may disable the automatic gain control system and further may direct the signal processing circuit 128 to hold the gain at a high level when operating with a high sample rate. For example, some embodiments of the powered ophthalmic lens 100 may support infrared signal detection only in environments with ambient light levels below 5000 lux and with infrared communication signals having incident power greater than 1 watt per square meter. The signal processing circuit 128 may operate with a gain dependent on the sample rate, an example of which is set forth subsequently. Under this range of conditions it may be possible to provide the data signal 136 with sufficient signal-to-noise ratio for detection while configuring the photodetector 126 and signal processing circuit 128 to have a constant gain from incident light energy to the amplitude or value of the data signal 136. In this way the system complexity may be minimized compared to a system that may operate with variable gain during infrared communication signal detection or processing.

In some embodiments, the signal processing circuit 128 may define at least a portion of the discrete signal path, as described herein. The signal processing circuit 128 may be implemented as a system comprising an integrating sampler, an analog to digital converter and a digital logic circuit configured to provide a digital data signal 136 based on the light-based signal 134. The system controller 130 also may be implemented as a digital logic circuit and implemented as a separate component or integrated with signal processing circuit 128. Portions of the signal processing circuit 128 and system controller 130 may be implemented in custom logic, reprogrammable logic or one or more microcontrollers as are well known to those of ordinary skill in the art. The signal processing circuit 128 and system controller 130 may comprise associated memory to maintain a history of values of the light-based signal 134, the data signal 136 or the state of the system. Any suitable arrangement and/or configuration may be utilized.

The power source 102 supplies power for numerous components comprising the ophthalmic lens 100. The power may be supplied from a battery, energy harvester, or other suitable means as is known to one of ordinary skill in the art. Essentially, any type of power source 102 may be utilized to provide reliable power for all other components of the system. A blink sequence or an infrared communication signal having a predetermined sequence or data message value may be utilized to change the state of the system and/or the system controller as set forth above. Furthermore, the system controller 130 may control other aspects of a powered ophthalmic lens depending on input from the signal processing circuit 128, for example, changing the focus or refractive power of an electronically controlled lens through the system controller 130. As illustrated, the power source 102 is coupled to each of the other components through the power management circuit 104 and would be connected to any additional element or functional block requiring power. The power management circuit 104 may comprise electronic circuitry such as switches, voltage regulators or voltage charge pumps to provide voltage or current signals to the functional blocks in the ophthalmic lens 100. The power management circuit 104 may be configured to send or receive control signals to or from the system controller 130. For example, the system controller 130 may direct the power management circuit 104 to enable a voltage charge pump to drive the actuator 112 with a voltage higher than that provided by the power source 102.

The actuator 112 may comprise any suitable device for implementing a specific action based upon a received command signal. For example if a blink activation sequence is detected, as described above, the system controller 110, 130 may enable the actuator 112 to control a variable-optic element of an electronic or powered lens. The actuator 112 may comprise an electrical device, a mechanical device, a magnetic device, or any combination thereof. The actuator 112 receives a signal from the system controller 110, 130 in addition to power from the power source 102 and the power management circuit 104 and produces some action based on the signal from the system controller 110, 130. For example, if the system controller 110, 130 detects a signal indicative of the wearer trying to focus on a near object, the actuator 112 may be utilized to change the refractive power of the electronic ophthalmic lens, for example, via a dynamic multi-liquid optic zone. In an alternate exemplary embodiment, the system controller 110, 130 may output a signal indicating that a therapeutic agent should be delivered to the eye(s). In this exemplary embodiment, the actuator 112 may comprise a pump and reservoir, for example, a microelectromechanical system (MEMS) pump. As set forth above, the powered lens of the present disclosure may provide various functionality; accordingly, one or more actuators 112 may be variously configured to implement the functionality. For example, a variable-focus ophthalmic optic or simply the variable-focus optic may be a liquid lens that changes focal properties, e.g. focal length, in response to an activation voltage applied across two electrical terminals of the variable-focus optic. It is important to note, however, that the variable-focus lens optic may comprise any suitable, controllable optic device such as a light-emitting diode or microelectromechanical system (MEMS) actuator.

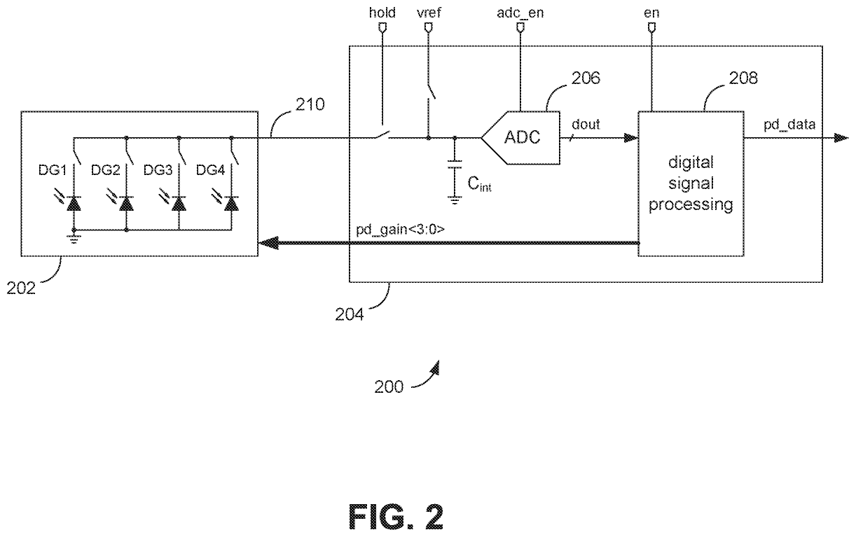

FIG. 2 illustrates, in party schematic and partly block-diagram form, a photodetection system 200 comprising a photodetector 202 and a signal processing circuit 204 in accordance with some embodiments of the present disclosure. The photodetection system 200 may define at least a portion of the discrete signal path, as described herein. The photodetector 202 may comprise photodiodes DG1, DG2, DG3 and DG4, which are selectively coupled to a cathode node 210. The signal processing circuit 204 may comprise an analog to digital converter 206 and a digital signal processing circuit 208. The analog to digital converter 206 may be configured to receive a signal from the photodetector 202 and to provide a digital converted signal (dout) to the digital signal processing circuit 208. The digital signal processing circuit 208 may comprise circuits configured to perform digital signal processing, including one or more of filtering, processing, detecting, and otherwise manipulating/processing data to permit incident light detection for downstream use. The digital signal processing circuit 208 may be configured to provide a gain setting signal pd_gain to the photodetector 202, for example to perform the selective coupling of photodiodes DG1, DG2, DG3 and DG4. The digital signal processing circuit 208 may be further configured to receive control signals to enable or disable switches, circuits or operating modes of circuits within the digital signal processing circuit 208.

In some embodiments of the present disclosure, signal processing circuit 204 may further comprise an integration capacitor and switches to selectively couple the cathode node 210 or a voltage reference to the integration capacitor. The integration capacitor may be configured to integrate a photocurrent developed by the photodetector 202 and to provide a voltage signal based on the integration time and a magnitude of the photocurrent. The photodetection system 200 may operate with a periodic sampling rate. The photodetection system 200 may operate with a predetermined sampling rate. The sampling rate may include a plurality of sampling rates and may vary depending on the signal or sequence being processed. During each sample interval the integration capacitor may be first coupled to a voltage reference, such that the integration capacitor is precharged at the start of the sample interval to a reference voltage, and then may be disconnected from the voltage reference and coupled to the cathode node 210 to integrate the photocurrent for an integration time corresponding to all or most of the remainder of the sample interval. The magnitude of the voltage signal at the end of the integration time is proportional to the integration time and the magnitude of the photocurrent. Shorter sample intervals corresponding to higher sample rates have lower voltage gain than longer sample intervals and lower sampling rates, where the voltage gain is defined as the ratio of the magnitude of the voltage signal at the end of the integration time to the magnitude of the photocurrent. At high sample rates more photodiodes may be coupled to cathode node 210 to increase the photocurrent to produce a higher magnitude voltage signal than would be produced with fewer diodes. Similarly, the number of photodiodes coupled to cathode node 210 may be increased or decreased in response to the magnitude of the photocurrent to ensure the magnitude of the voltage signal is within a useful dynamic range of the analog to digital converter 206. For example, an incident light energy of 1000 lux may generate a photocurrent of 10 pA in photodiode DG1. At a low sample rate of 0.1 s per sample or 10 Hz the photocurrent may be integrated on integration capacitor Cᵢₙₜ having a value of 5 picofarads (pF) for 0.1 s in turn providing a voltage of 200 mV on the integration capacitor Cᵢₙₜ and provided to the analog to digital converter 206. However a lower incident light energy of 200 lux will only generate 2 pA and an integrated voltage of 40 mV therefore leading to reduced signal dynamic range at the input to the analog to digital converter 206. Increasing the number of diodes by a factor of five, for example by coupling photodiode DG2 which may have a area four times that of photodiode DG1 provides a total photocurrent of 10 pA restoring the signal level to 200 mV at the input to the analog to digital converter 206. In a second example, an incident infrared light energy of 1 watt per square meter may generate a photocurrent of 3 pA total in photodiodes DG1 and DG2. At a 0.1 s sample rate and 0.1 s integration time this is sufficient to generate an integrated voltage of 60 mV. At a higher sample rate and shorter integration time of 390.625 ps or 2.56 kHz this photocurrent generates an integrated voltage of only 0.23 uV, which is too low for detection. Coupling photodiodes DG3 and DG4 provides larger total photodiode area and higher photocurrent on the order of 1.6 nA, leading to an integrated voltage of 125 mV, which provides significantly better signal level and dynamic range. The analog to digital converter 206 may be, for example, of a type that provides eight (8) bits of resolution in a full scale voltage range of 1.8V. For this example analog to digital converter signal levels from 40 mV to 200 mV yield digital output values between 5 and 28 with a maximum value of 255 for a 1.8V input signal. It will be appreciated by those of ordinary skill in the art that the photodiodes DG1, DG2, DG3 and DG4 may be designed to have any desirable scaling or areas for different purposes or system and environmental requirements, such as uniform weighting, binary weighting or other factors such as a factor of four in the preceding example.

FIG. 3 is a block diagram of digital detection logic 300 in accordance with some embodiments of the present disclosure. As an example, the digital detection logic may be comprised as the sequence detector 109, 129 (FIG. 1) or a portion thereof. The digital detection logic 300 may comprise a history shift register 302, a first comparator 304, and a second comparator 306. The digital detection logic 300 may define at least a portion of the discrete signal path, as described herein The detection logic 300 may be included and/or implemented as part of a system controller such as system controller 110, 130 (FIG. 1). The history shift register 302 may be configured to receive and store a predetermined number of digital values of a signal (pd_data signal) and to provide a history vector 308 comprising the sequence of stored values. The history shift register 302 may be further configured to selectively store the digital values in response to an external signal (pd_shift), which indicates to store a new value and discard an oldest value. The first comparator 304 may be configured to compare a first predetermined blink sequence to the history vector 308 and to indicate a successful match on an output blink detect (signal bl_det). The first comparator 304 may be further configured to receive a blink template vector (bl_tpl) indicative of the first predetermined blink sequence and a blink mask vector (bl_msk) indicative of samples or indices to selectively compare or ignore within the history vector 308. The second comparator 306 may configured to compare a second predetermined input sequence to the history vector 308 and to indicate a successful match on an output IR detect signal (ir_det). Although IR is referenced, it is understood that other wavelengths of input light may be used and detected by the photodetector. The second comparator 306 may be further configured to receive an ir template (vector ir_tpl) indicative of the second predetermined blink sequence (or representation of a clink sequence) and an ir mask vector (ir_msk) indicative of samples or indices to selectively compare or ignore within the history vector 308. The digital detection logic 300 may define at least a portion of the discrete signal path, as described herein. The digital detection logic 300 may be incorporated into a system controller of the type described above in accordance with the present disclosure.

The digital detection logic 300 may be configured to implement a blink detection algorithm, as described herein, and may also be configured to detect non-human-capable light sequences or patterns such as computer-generated communication signals. For example, a special light sequence may define the non-human-capable communication sequence or a portion thereof and may be caused to be transmitted to an eye of a wearer and may represent a pattern of alternating high and low light levels that have a frequency beyond a human-capable threshold for blinking. In some embodiments the special light sequence may comprise a number of, for example six, alternating high and low intervals of 0.2 seconds each. Such a sequence would be very unlikely to be produced by a human eye lid, and thus represents a unique sequence not produced by blinking. The special light sequence may be a programmable sequence and may be used as a trigger signal or preamble to indicate presence of or starting of an embedded data message. Although the term “non-human-capable” is used to differentiate signals from those that may be attributed to typical human-capable blink patterns, such non-human-capable sequences may be any pattern. Such non-human-capable sequences may have a frequency, duration, and/or complexity that is pre-defined to distinguish itself from human-capable blink patterns. As such, the systems described herein may be configured to determine the presence or absence of a human-capable blink pattern and a non-human-capable communication sequence using the same discrete processing path and/or control system.

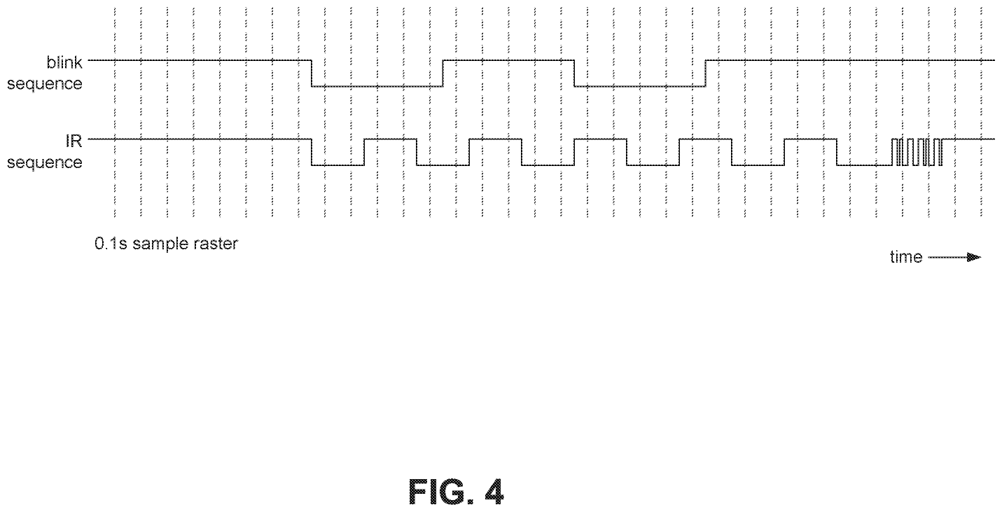

FIG. 4 illustrates blink and IR sequences (e.g., non-human-capable communication sequences) in accordance with some embodiments of the present disclosure. The light levels for each sequence are plotted versus time with a vertical level having arbitrary units indicating light energy incident on a photodetector. In some embodiments the blink sequence may comprise two low intervals of 0.5 seconds separated by a high interval of 0.5 seconds. A template of length 24 of data values representative of the blink sequence sampled at a 0.1 second or 10 Hz rate is [1, 1, 1, 1, 1, 1, 0, 0, 0, 0, 0, 1, 1, 1, 1, 1, 0, 0, 0, 0, 0, 1, 1, 1]. In some embodiments the IR sequence may comprise a number of, for example six, alternating high and low intervals of 0.2 seconds each. Such a sequence would be very unlikely to be produced by a human eye lid, and thus represents a unique sequence not produced by blinking. At the end of the IR sequence is illustrated a dense alternation of signal values representative of a higher data rate communication signal. In the present disclosure the special IR sequence indicates that a higher data rate IR data message is starting. A template of length 24 of data values representative of the IR sequence sampled at a 0.1 second or 10 Hz rate is [1, 1, 0, 0, 1, 1, 0, 0, 1, 1, 0, 0, 1, 1, 0, 0, 1, 1, 0, 0, 1, 1, 0, 0].

As discussed herein, the special IR sequence of the non-human-capable pattern may indicate the presence of or starting of a higher data rate IR communication signal containing a communication data message (e.g., signal). In some embodiments the higher data rate IR communication signal may include a preamble of 40 alternating Manchester 0, 1 symbols (0x55555, 0x55555), which may be equivalent to 20 pulses. Other preambles and non-human-capable communication sequences may be used. The communication message may include a synchronization word or sync word (e.g., device address: 7 bits with value 0x59), a register address of 8 bits, a register data of 8 bits, and a parity bit. Other bit lengths for each component of the communication message may be used. In some embodiments the communication message may be transmitted on a repeated pattern within a predetermined timeframe (e.g., timeout). As such, the configuration of the communication message facilitates the data within the message to be detected and extracted for decoding and/or retransmission, as will be discussed in further detail below.

Frame synchronization is a process through which incoming frame alignment signals, for example, distinctive symbol or bit sequences, are identified and distinguished from data, thereby permitting the data within a stream of framed data to be extracted for decoding and/or retransmission. The frame structure provides a message frame comprising a transmit synchronization word, sync, and a data word. In some exemplary embodiments, the data word may comprise a device address of the intended receiver, addr, and a command word, cmd, to provide an instruction or information to the receiver. In some exemplary embodiments, the data word may comprise a register address of an interested register to modify in the receiver and a new register data value. In some embodiments rather than a long preamble during which the receiver must wait for the transmitted data, the sync, addr and cmd words are sent repeatedly for the full frame interval. The receiver may then be on for only the time required to detect the sync word and decode the address and command. Since the sync, addr and cmd words are typically much shorter than the receiver strobe interval, Trx-strobe, the receiver on time and average power are greatly reduced relative to prior art asynchronous communication protocols. As illustrated, the transmit time Ttx is set to be greater than the receiver strobe interval Trx-strobe.

Because a receiver may begin decoding transmitted data at any given point, the synchronization word, sync, must be uniquely detected. Prior art communication protocols employ Block Codes, i.e. error correcting codes that encode data in blocks, with code words, e.g., allowable words for sync, addr and cmd or other message data, that are not unique when shifted and/or rotated left or right. The use of this type coding would lead to false detection of the synchronization word when it is offset within the frame.

In accordance with an exemplary embodiment of the present disclosure, the synchronization words may be selected to be an Orthogonal Cyclic Code, such as a Gold code or Gold code sequence, which is unique regardless of the shift or starting point for decoding relative to other Gold code sequences of the same length. In this exemplary embodiment, the address and command words are also selected or limited such that the message frame does not match the synchronization word at any shift. In an alternate exemplary embodiment, the allowable list or code book of address and command words may be selected to minimize the correlation of address and command words to the synchronization word, as may be characterized by the cross-correlation or the Hamming Distance as is known in the relevant art. In yet another alternate exemplary embodiment, the address and command word set may be selected only from a set of Gold codes or Gold sequences to minimize the cross-correlation to the synchronization word.

The generation of Gold codes or sequences is known in the relevant art. Gold codes or sequences are generated from two pseudo-random sequence generators having preferred polynomials. Preferred polynomials are those that lead to maximal length sequences (m-sequences, length=2m−1), and that have cross correlation values of {1, t}, where t=2(m+1)/2+1 or 2(m+2)/2+1 for odd or even m. Gold codes are available only in certain lengths, which constrains their use somewhat for short code words. It is important to note that while Gold codes may have the best cross-correlation properties, other code words may be utilized which have reasonably high distances to the Gold codes. Accordingly, in another exemplary embodiment, these other code words with good (low) cross-correlation may be utilized for device addresses and commands while the Gold codes may be utilized as synchronization words.

In yet another alternate exemplary embodiment, the synchronization, address and command words may be selected as set forth in the process described below. In the first step of the exemplary process, an address length, LA, is selected or chosen to provide more than a desired number of distinct addresses for a particular application. For example, fifteen (15) million addresses may be desired for a particular application. Accordingly, for fifteen (15) million addresses, the required address length is twenty-four (24) bits because twenty-four bits yields over sixteen (16) million unique addresses (224=16,777,216) and twenty-three (23) bits yields only over eight (8) million addresses. In the second step of the exemplary process, a command length, LC, is selected or chosen to provide a desired number of distinct commands. For example, eight (8) commands may be desired for the particular application. Accordingly, for eight (8) commands, the required command length is three (3) bits because three bits yield eight (8) commands (23=8). In the third step of the exemplary process, the synchronization word is selected from a set of Gold codes with a length close to that of the combined address and command word length. For a Gold code, the word length is 2m−1; accordingly, for m=1, the word length is one (1) bit, for m=2, the word length is three (3) bits, for m=3, the word length is seven (7) bits, for m=4, the word length is fifteen (15) bits and for m=5, the word length is thirty-one (31) bits. The longer the synchronization word, the lower the number of synchronization+address+command combinations that will contain a match to the synchronization word at some offset. Accordingly, any address from the list of allowable addresses that leads to matches at some offsets is removed; however, this selection is a tradeoff between overall message length, and corresponding receiver on time, versus the total number of remaining addresses. In this example, a synchronization word length of fifteen (15) bits is good enough to retain most of the possible addresses as is explained in more detail subsequently. Also for the synchronization word, if one is utilizing a non-return to zone (NRZ) symbol format, it is generally advantageous if the average value of the symbols is a value of one-half. This can help with determining where the threshold value should be on a comparator in a signal processing portion of the receiver. In embodiments utilizing Manchester coding, which provides an average value of 0.5 for each symbol, this is less of a concern. Accordingly, in this example, the fifteen (15) bit synchronization word is selected to be 100110010101101, which comprises eight l's and seven 0's for an average value of 0.533. In the fourth and final step of the exemplary process, a useable set of addresses is determined by constructing all possible sequences of synchronization word, address word and command word, determining the possible sample sequences of length LS formed by taking subsets of the synchronization+address+command+synchronization sequence minus one symbol starting at each possible offset, and removing those addresses that have a strong correlation, for example, a perfect match or small Hamming Distance, to the synchronization word at some offsets. In this example which utilizes a twenty-four (24) bit address length, a three (3) bit command length, and a fifteen (15) bits Gold code of 100110010101101, implementing the search of step four of the exemplary process results in 69,632 addresses out of the 16,777,216 possible addresses that yield sequences which match the synchronization word at some offsets. Thus, only a relatively small subset of the possible addresses must be removed from the set of possible addresses.

It is important to note that those of ordinary skill in the relevant art will recognize that the synchronization word may be chosen or selected in any suitable manner, including utilizing a random number generator and address and command words chosen to avoid a strong correlation. It is also important to note that the length of the synchronization word, the address word and the command word may be selected to suit the needs of a particular system. For example, very short word lengths may be used in a system that only requires a small number of receivers to minimize receiver on time. Similarly, much longer synchronization address and command words may be chosen to support a much larger number of users or commands.

Modulation is the technique of adding the message signal to some form of carrier signal. In other words, modulation involves varying one or more properties of a high frequency, periodic waveform, the carrier signal, with a modulating signal that comprises the data or information to be transmitted. There are analog modulation methods, including amplitude modulation, frequency modulation and phase modulation, and there are digital modulation methods, including phase-shift keying, frequency-shift keying, amplitude-shift keying and quadrature amplitude modulation. As the present disclosure is a digital-based system, digital modulation techniques as set forth herein may be utilized. Some exemplary embodiments of the present disclosure may utilize on-off keying to modulate the amplitude of a carrier signal. The carrier signal may be a radio frequency electromagnetic signal or a visible or infrared light signal, such as that emitted from a light-emitting diode. The modulated signal is transmitted, detected and demodulated at the other end of the communication channel; namely, the receiver. Essentially, modulation techniques deal with how the data signal is incorporated onto a carrier signal, but do not deal with how the data signal is created from the data or information to be transmitted. Coding is a technique through which a message or data signal is constructed from the data or information to be communicated. Coding techniques include NRZ coding, BiPhase coding and Manchester coding. Coding may be considered an additional function of the digital modulator.

Manchester coding is a common data coding technique. Manchester coding provides for adding the data rate clock to the data or information to be utilized on the receiving end of the communication channel. Manchester encoding is the process of adding the correct transitions to the message signal in relation to the data or information that is to be transmitted over the communication channel.

A “symbol” is one unit of information sent over a communication channel. The value of the symbol is determined, in the current disclosure, by the voltages on the communication channel at different times. The “symbol time” is simply the duration of the symbol. The “symbol rate” is the reciprocal of the symbol time, expressed in symbols per second. Each symbol may represent one bit of the binary data stream or a multi-bit value. For Manchester encoded symbols, there are two possible voltage levels, high or low, and each symbol comprises one voltage level for the first half of the symbol time and the other voltage level for the second half of the symbol time. In accordance with the present disclosure, the convention utilized is that the voltage level in the first half symbol time defines the value of the symbol. This is explained in detail subsequently. Manchester data always has a mid-symbol transition even if the symbol values are constant for a long time or if they are changing. In addition, there might not be transitions in the signal levels from the end of one symbol to the beginning of the next, for example, a 0 to a 1 symbol will have a high voltage level at the end of the 0 and start of the 1 symbol, but there is always a mid-symbol transition. A “sample” is a captured or recorded value from an instant in time or from a small window in time. In accordance with the present disclosure, the incoming signal is periodically sampled and from the value of each sample, the value of the current symbol is determined. The rate of periodic sampling is the “sampling rate”. For Manchester decoding, the incoming signal is “oversampled,” meaning that a sampling rate that is greater than the symbol rate by at least a factor of 2× is utilized. In the present disclosure, 8× oversampling is utilized. Because the symbol value is determined by the voltage level in the first half symbol time, one only needs to sample in the first half of the symbol time. Accordingly, sampling may be stopped and power saved for some finite time.

In accordance with an exemplary embodiment of the disclosure, Manchester coding is utilized. In Manchester coding, the transmit symbols are split into two parts, one having a 0 value and the other having a 1 value. For example, if the first half of the symbol is a 0 and the second half is a 1, then this is a 0 symbol, whereas if the first half of the symbol is a 1 and the second half of the symbol is a 0, then this is a 1 signal. Thus each transmittal symbol has a center-of-symbol transition or edge and these transitions may be detected with each symbol regardless of the sequence of data bits or symbols being transmitted.

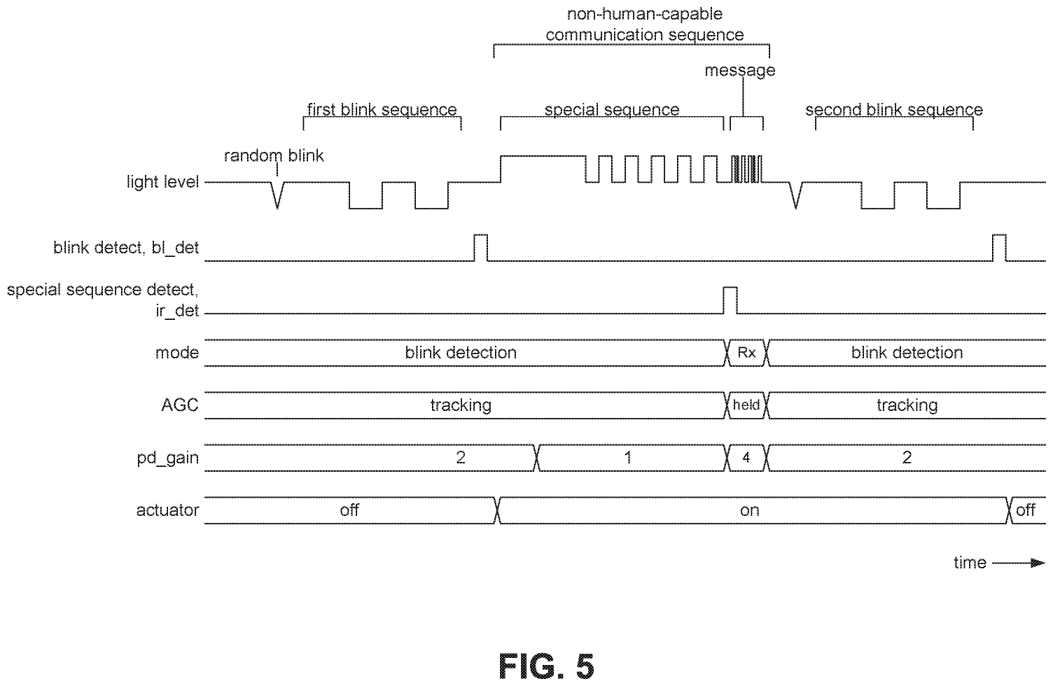

FIG. 5 illustrates a timing diagram of an operational sequence of a combined blink detection and communication system in accordance with some embodiments of the present disclosure. A light level received by a photodetector is illustrated in the first signal labeled “light level.” As indicated the light level may vary with random blinks. Over time the light level varies with a first blink sequence, an IR special sequence, an IR message, and a second blink sequence. One or more of the IR special sequence and the IR message may define at least a portion of a non-human-capable communication sequence. The light level may increase during the IR special sequence and/or IR message of the non-human-capable communication sequence because the signals may be produced by a light source such as an infrared light-emitting diode (IR LED) that is in addition to the ambient light level. The second signal labeled “blink detect, bl_det” illustrates the output of a digital detection logic circuit indicating that a predetermined blink sequence has been detected. The third signal labeled “special sequence detect, ir_det” illustrates the output of a digital detection logic circuit indicating that a predetermined special sequence (e.g., IR sequence) has been detected. The special sequence may define at least a portion of a non-human-capable communication sequence. The fourth signal labeled “mode” illustrates an operating mode of the combined blink detection and communication system, corresponding to a state of a system controller, and being in either a blink detection mode or a communication receive mode labeled “Rx.” The fifth signal labeled “AGC” illustrates the state of an automatic gain control function in accordance with some embodiments of the present disclosure. In this illustration the automatic gain control is in a tracking mode during blink detection, such that a gain of a photodetector is varied in response to changes in ambient light level, and is in a held mode during IR communication reception. The sixth signal labeled “pd_gain” illustrates a photodetector gain setting that varies in response to the received light level. The gain setting varies from a medium value of 2 with ambient light levels, to a low value of 1 with additional light energy from the IR special sequence, to a high value of 4 during communication reception, and back to a medium value of two with ambient light levels after the IR message ends. As described previously the pd_gain signal may determine the number of photodiodes selectively coupled to provide photocurrent to the signal processing circuit. In the ambient and ambient plus IR conditions the signal processing circuit in this example varies the pd_gain value to maintain a signal level within a desired dynamic range, such as between a range of output digital values of 30 and 220 for an eight bit analog to digital converter. As in previous examples, the total light energy increases from ambient to ambient plus IR during the initial special sequence portion of the IR communication signal (e.g., non-human-capable pattern), and the signal processor reduces the gain of the photodetector, in this case from a value of 2 to a value of 1, to maintain the signal within a desired range. During the IR communication message (e.g., message) the system controller may increase the sample rate of the photodetection system and also increases the pd_gain value, in this example to a value of 4, and therefore the number of photodiodes coupled to provide photocurrent in order to maintain the signal within the desired range when operating at the higher sample rate and lower integration time. It will be appreciated by those of ordinary skill in the art that other types of photodetector systems may require or may be adapted to different types of gain control. The seventh and last signal labeled “actuator” illustrates the state of an actuator that is controlled in response to the detection of blink sequences. In this example the actuator starts in an off state, is turned on after detection of the first blink sequence and is turned off again after detection of the second blink sequence.

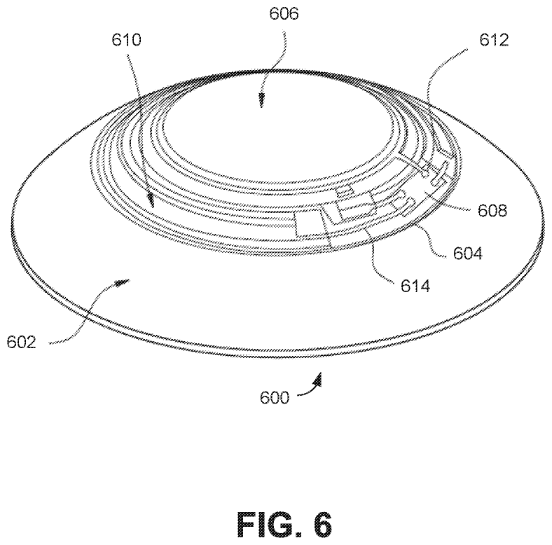

FIG. 6 is a diagrammatic representation of an exemplary electronic insert, including a combined blink detection and communication system, positioned in a powered or electronic ophthalmic device in accordance with the present disclosure. As shown, a contact lens 600 comprises a soft plastic portion 602 which comprises an electronic insert 604. This insert 604 includes a lens 606 which is activated by the electronics, for example, focusing near or far depending on activation. Integrated circuit 608 mounts onto the insert 604 and connects to batteries 610, lens 606, and other components as necessary for the system. The integrated circuit 608 includes a photodetector 612 and associated photodetector signal path circuits. The photodetector 612 may comprise an array of photodiodes and faces outward through the lens insert and away from the eye, and is thus able to receive ambient light. The photodetector 612 may be implemented on the integrated circuit 608 (as shown) for example as a single photodiode or array of photodiodes. The photodetector 612 may also be implemented as a separate device mounted on the insert 604 and connected with wiring traces 614. When the eyelid closes, the lens insert 604 including 5 photodetector 612 is covered, thereby reducing the light level incident on the photodetector 612. The photodetector 612 is able to produce a photocurrent in response to the level of ambient light and/or infrared light.

The second signal path may be discrete from the first signal path. For example, the second signal path may be independent of the first signal path such that the non-human-capable communication sequence is determined independent of the first signal path. Sampling (e.g., or otherwise accessing) the first signal path may comprise sampling a first communication channel. Sampling (e.g., or otherwise accessing) the second signal path may comprise sampling a second communication channel. The first communication channel may comprise a first wavelength band, frequency channel, time divided channel, and/or the like. The second communication channel may comprise a second wavelength band, frequency channel, time divided channel, and/or the like. The first wavelength band may visible light. The second communication channel may comprise infrared light. In some scenarios, the ophthalmic device may comprise multiple photodetectors. The first communication channel may be sampled by a first photodetector. The second communication channel may be sampled by a second photodetector. Data and/or signals on the first communication channel (e.g., or first signal path) may be used to trigger sampling, analysis, on the second communication channel (e.g., second signal path). Data detected on one signal path or channel may be used to trigger sampling and/or analysis for other data on the same signal path or channel or a different signal path or channel. As an example, the human-capable blink pattern may comprise an involuntary blink pattern. Determining the involuntary blink pattern (e.g., or other pattern) on the first signal path may be used to trigger sampling via the second signal path (e.g., or analysis for non-human-capable patterns on the same or a different channel or signal path).

Although shown and described in what is believed to be the most practical and preferred embodiments, it is apparent that departures from specific designs and methods described and shown will suggest themselves to those skilled in the art and may be used without departing from the spirit and scope of the disclosure. The present disclosure is not restricted to the particular constructions described and illustrated, but should be constructed to cohere with all modifications that may fall within the scope of the appended claims.

What is claimed is:

1. A method for detecting signal patterns, the method comprising:
sampling, via a first signal path disposed on an ophthalmic device, light incident on an eye of an individual and at least temporarily saving first collected samples;
sampling, via a second signal path disposed on an ophthalmic device, light incident on an eye of an individual and at least temporarily saving second collected samples, wherein the second signal path is discrete from the first signal path;
analyzing the first collected samples to determine the existence or absence of a human-capable blink pattern;
analyzing the second collected samples to determine the existence or absence of a non-human-capable communication sequence; and
providing an indication signal to activate and control one or more properties of the ophthalmic device based at least on one or more of the existence or absence of the human-capable blink pattern and the existence or absence of the non-human-capable communication sequence.

2. The method of claim 1, wherein the ophthalmic device comprises a wearable lens.

3. The method of claim 1, wherein the ophthalmic device comprises a contact lens or an implantable lens, or a combination of both.

4. The method of claim 1, further comprising, upon determining the existence of the human-capable blink pattern:
determining, from the first collected samples, when an eyelid is open or closed in order to characterize blinking by calculating a number, time period and pulse width of one or more of a plurality of blinks;
comparing a number of blinks in a given time period, durations of the blinks in the given time period, and a time between blinks in the given time period to a stored set of samples representative of one or more predetermined blink sequences to determine patterns in blinking; and
determining if the blinks correspond to one or more of a predetermined blink sequences, the step of determining including allowance for deviations in the durations of the blinks from the durations of the blinks in the one or more predetermined blink sequences; and
utilizing the one or more blink sequences as the indication signal to activate and control the one or more properties of the ophthalmic device.

5. The method of claim 1, further comprising, upon determining the existence of the human-capable blink pattern:
determining when an eyelid is open or closed in order to divine the number, time period, and pulse width of the blinks from the first collected samples;
comparing the number of blinks in a given time period, durations of the blinks in the given time period, and the time between blinks in the given time period to a stored set of samples representative of one or more predetermined intentional blink sequences to determine patterns in blinking; and
determining if the blinks correspond to one or more of the predetermined intentional blink sequences.

6. The method of claim 1, wherein the sampling is conducted at a predetermined sampling rate.

7. The method of claim 1, wherein the sampling is conducted over a plurality of sampling rates.

8. The method of claim 1, wherein the non-human capable communication sequence comprises one or more of a message or a special sequence indicative of the presence of the message.

9. The method of claim 8, wherein the special sequence comprises two or more time intervals having a high light level separated by a time interval having a low light level wherein the durations of the intervals are at most 0.2 seconds.

10. The method of claim 1, wherein the existence of a non-human-capable communication sequence is determined, and wherein the non-human-capable communication sequence comprises a preamble having at least 10 alternating Manchester symbols.

11. The method of claim 1, wherein the existence of a non-human-capable communication sequence is determined, and wherein the non-human-capable communication sequence comprises a synchronization word.

12. The method of claim 1, wherein the existence of a non-human-capable communication sequence is determined, and wherein the non-human-capable communication sequence comprises a register address and register data.

13. The method of claim 1, wherein the existence of a non-human-capable communication sequence is determined, and wherein the non-human-capable communication sequence comprises a register word and a data word.

14. The method of claim 1, wherein the existence of a non-human-capable communication sequence is determined, and wherein the non-human-capable communication sequence comprises a synchronization word, an address word, and a data word.

15. The method of claim 1, wherein the second signal path being discrete from the first signal path comprises the second signal path being independent of the first signal path such that the non-human-capable communication sequence is determined independent of the first signal path.

16. The method of claim 1, wherein the human-capable blink pattern comprises an involuntary blink pattern, and wherein determining the involuntary blink pattern on the first signal path is used to trigger sampling via the second signal path.

17. The method of claim 1, wherein sampling via the first signal path comprises sampling a first communication channel and sampling the second signal path comprising sampling a second communication channel.

18. The method of claim 17, where the first communication channel comprises a first wavelength band and the second communication channel comprise a second wavelength band.

19. The method of claim 18, wherein the first wavelength band comprises visible light and the second communication channel comprises infrared light.

20. The method of claim 17, wherein the first communication channel is sampled by a first photodetector and the second communication channel is sampled by a second photodetector.

21. A system comprising:
a light detector configured to output a signal corresponding to the intensity of light incident upon an eye; and
a processor configured to receive the signal, wherein the processor defines at least a portion of the discrete signal path, and wherein the processor is configured to:
sample, via the discrete signal, light incident on an eye of an individual and at least temporarily saving collected samples;
analyze the collected samples to determine the existence or absence of a human-capable blink pattern;
analyze the collected samples to determine the existence or absence of a non-human-capable communication sequence; and
cause provision of an indication signal to activate and control one or more properties of the electronic ophthalmic device based at least on one or more of the existence or absence of the human-capable blink pattern and the existence or absence of the non-human-capable communication sequence.

22. The system of claim 21, wherein the ophthalmic device comprises a wearable lens.

23. The system of claim 21, wherein the ophthalmic device comprises a contact lens or an implantable lens, or a combination of both.

24. The system of claim 21, wherein the processor is further configured to:
upon determining the existence of the human-capable blink pattern: determine when an eyelid is open or closed in order to characterize blinking by calculating a number, time period and pulse width of one or more of a plurality of blinks from the collected samples;
comparing a number of blinks in a given time period, durations of the blinks in the given time period, and a time between blinks in the given time period to a stored set of samples representative of one or more predetermined intentional blink sequences to determine patterns in blinking; and
determine if the blinks correspond to one or more of a predetermined intentional blink sequences, the step of determining including allowance for deviations in the durations of the blinks from the durations of the blinks in the one or more predetermined intentional blink sequences; and
utilize the one or more intentional blink sequences as the indication signal to activate and control the one or more properties of the ophthalmic device.

25. The system of claim 21, wherein the processor is further configured to:
upon determining the existence of the human-capable blink pattern:
determine when an eyelid is open or closed in order to divine the number, time period, and pulse width of the blinks from the collected samples;
comparing the number of blinks in a given time period, durations of the blinks in the given time period, and the time between blinks in the given time period to a stored set of samples representative of one or more predetermined intentional blink sequences to determine patterns in blinking; and
determine if the blinks correspond to one or more of the predetermined intentional blink sequences.

26. The system of claim 21, wherein the sampling is conducted at a predetermined sampling rate.

27. The system of claim 21, wherein the sampling is conducted over a plurality of sampling rates.

28. The system of claim 21, wherein the non-human capable communication sequence comprises a message and a special sequence indicative of the presence of the message.

29. The system of claim 28, wherein the special sequence comprises two or more time intervals having a high light level separated by a time interval having a low light level wherein the durations of the intervals are at most 0.2 seconds.

30. The system of claim 21, wherein the existence of a non-human-capable communication sequence is determined, and wherein the non-human-capable communication sequence comprises a preamble having at least 10 alternating Manchester symbols.

31. The system of claim 21, wherein the existence of a non-human-capable communication sequence is determined, and wherein the non-human-capable communication sequence comprises a synchronization word.

32. The system of claim 21, wherein the existence of a non-human-capable communication sequence is determined, and wherein the non-human-capable communication sequence comprises a register address and register data.

33. The system of claim 21, wherein the existence of a non-human-capable communication sequence is determined, and wherein the non-human-capable communication sequence comprises a register word and a data word.

34. The system of claim 21, wherein the existence of a non-human-capable communication sequence is determined, and wherein the non-human-capable communication sequence comprises a synchronization word, an address word, and a data word.

35. The method of claim 21, wherein the human-capable blink pattern comprises an involuntary blink pattern, and wherein determining the existence of the involuntary blink pattern on the first signal path is used to trigger analyzing the collected samples to determine the existence or absence of a non-human-capable communication sequence.

36. A method for detecting signal patterns, the method comprising:
sampling, via a first signal path disposed on an ophthalmic device that fits on or in an eye of a user, light incident on an eye of an individual and at least temporarily saving collected samples;
analyzing, by a controller and via the first signal path, the collected samples to determine the existence of a non-human-capable blink pattern;
triggering, based on the existence of the non-human-capable blink pattern, a second signal path to enable analysis of the collected samples to determine a sequence indicative of an embedded communication message; and
providing an indication signal to a control system to activate and control one or more properties of the ophthalmic device based at least on the embedded communication message.

37. The method of claim 36, wherein the ophthalmic device comprises a wearable lens.

38. The method of claim 36, wherein the ophthalmic device comprises a contact lens or an implantable lens, or a combination of both.

39. The method of claim 36, wherein the sampling is conducted at a predetermined sampling rate.

40. The method of claim 36, wherein the sampling is conducted over a plurality of sampling rates.

41. The method of claim 40, wherein the sequence indicative of an embedded communication message comprises two or more time intervals having a high light level separated by a time interval having a low light level wherein the durations of the intervals are at most 0.2 seconds.

42. The method of claim 36, wherein the sequence indicative of an embedded communication message comprises a preamble having at least 10 alternating Manchester symbols.

43. The method of claim 36, wherein the communication message comprises synchronization word.

44. The method of claim 36, wherein the communication message comprises a register address and register data.

45. The method of claim 36, wherein the communication message comprises a register word and a data word.

46. The method of claim 36, wherein the communication message comprises a synchronization word, an address word, and a data word.

47. The method of claim 36, wherein the second signal path is independent of the first signal path such that the sequence is determined independent of the first signal path.

48. The method of claim 36, wherein determining the sequence indicative of the embedded communication message comprises determining an involuntary blink pattern, and wherein determining the involuntary blink pattern is used to trigger detection on the second signal path.

49. A method for detecting signal patterns, the method comprising:
sampling, via a first signal path disposed on an ophthalmic device that fits on or in an eye of a user, light incident on an eye of an individual and at least temporarily saving collected samples;
analyzing, by a controller and via the first signal path, the collected samples to determine the existence or absence of a special sequence indicative of the presence of an embedded communication message, wherein the first single path is configured to determine the existence or absence of a human-capable blink pattern;
energizing, based on the existence of the special sequence, a second signal path to enable analysis of the collected samples to determine the embedded communication message; and
providing an indication signal to a control system to activate and control one or more properties of the ophthalmic device based at least on the embedded communication message.

50. The method of claim 49, wherein the ophthalmic device comprises a wearable lens.

51. The method of claim 49, wherein the ophthalmic device comprises a contact lens or an implantable lens, or a combination of both.

52. The method of claim 49, wherein the sampling is conducted at a predetermined sampling rate.

53. The method of claim 49, wherein the sampling is conducted over a plurality of sampling rates.

54. The method of claim 49, wherein the sequence indicative of an embedded communication message comprises two or more time intervals having a high light level separated by a time interval having a low light level wherein the durations of the intervals are at most 0.2 seconds.

55. The method of claim 49, wherein the sequence indicative of an embedded communication message comprises a preamble having at least 10 alternating Manchester symbols.

56. The method of claim 49, wherein the communication message comprises synchronization word.

57. The method of claim 49, wherein the communication message comprises a register address and register data.

58. The method of claim 49, wherein the communication message comprises a register word and a data word.

59. The method of claim 49, wherein the communication message comprises a synchronization word, an address word, and a data word.

60. The method of claim 49, wherein the second signal path is independent of the first signal path such that the sequence is determined independent of the first signal path.

61. The method of claim 49, wherein determining the sequence indicative of the embedded communication message comprises determining an involuntary blink pattern on the first signal path, and wherein the second signal path is energized based on determining the involuntary blink pattern.
